(19) United States
(12) Patent Application Publication
(10) Pub. No.: US 2008/0287935 A1
(43) Pub. Date: Nov. 20, 2008

(54) SYSTEM AND METHOD FOR PHOTOABLATION USING MULTIPLE FOCAL POINTS USING CYCLICAL PHASE MODULATION

(76) Inventor: Josef Bille, Heidelberg (DE)

Correspondence Address: NYDEGGER & ASSOCIATES, 348 OLIVE STREET, SAN DIEGO, CA, 92103, US

(21) Appl. No.: 11/743,564

(22) Filed: May 2, 2007

Related U.S. Application Data
(63) Continuation-in-part of application No. 11/682,976, filed on Mar. 7, 2007.
Application No. 11/682,976 is a continuation-in-part of application No. 11/190,052, filed on Jul. 26, 2005.
Application No. 11/190,052 is a continuation-in-part of application No. 11/033,967, filed on Jan. 12, 2005, now Pat. No. 7,232,436.
Application No. 11/033,967 is a continuation-in-part of application No. 10/293,226, filed on Nov. 13, 2002, now Pat. No. 6,887,232.

(51) Int. Cl. A61B 18/20

(52) U.S. Cl. 606/11

(57) ABSTRACT

A system and method for performing ophthalmic laser surgery requires directing a laser beam through a stationary beam splitter to create a pattern of multi-focal spots. Also, a beam scanner is used to move this pattern along a substantially spiral path in a target area of tissue. To compensate for cyclical changes in orientation of the pattern relative to its spiral path, a computer is used to phase modulate pattern movement. Specifically, this phase modulation is expressed as:

v′=v(1+F sin(nθ))

where v is a variable (e.g. angular speed, line spacing, or z-spacing), v′ is the phase modulated variable, F is a magnitude factor for phase modulation control, n is an integer, and θ is an angular position of the pattern during phase modulation.

This application is a continuation-in-part of pending application Ser. No. 11/682,976 filed Mar. 7, 2007, which is a continuation-in-part of pending application Ser. No. 11/190,052 filed Jul. 26, 2005, which is a continuation-in-part of pending application Ser. No. 11/033,967 filed Jan. 12, 2005, which is a continuation-in-part of application Ser. No. 10/293,226, filed Nov. 13, 2002, which issued as U.S. Pat. No. 6,887,232 on May 3, 2005. The contents of application Ser. Nos. 11/682,976, 11/190,052 and 11/033,967, and issued U.S. Pat. No. 6,887,232, are incorporated herein by reference.

### FIELD OF THE INVENTION

The present invention pertains generally to systems and methods for performing ophthalmic laser surgery. More particularly, the present invention pertains to systems and methods for performing ophthalmic laser surgery using multiple focal spots. The present invention is particularly, but not exclusively, useful as a system and method for moving a pattern of multiple focal spots through a target area of tissue in accordance with a phase modulated routine.

### BACKGROUND OF THE INVENTION

Laser Induced Optical Breakdown (LIOB) of corneal tissue is typically accomplished by focusing a laser beam to a very small focal spot in the tissue that is to be photoablated. During an ophthalmic laser surgical procedure, after LIOB has been accomplished at one location, the focal spot is moved along a predetermined path, through a predetermined distance (i.e. ten microns), and LIOB is again accomplished. A sequence of such movements follows until all tissue in the target area has been effectively subjected to LIOB.

For ophthalmic laser surgery, the time duration of a procedure can be of the utmost importance. Stated differently, it is very desirable to perform a procedure in the shortest possible time. Still, LIOB of the tissue must be effective, and the results must be efficacious. To do this, there are essentially two different approaches that can be followed. One is to move the laser spot faster. The other is to create a plurality of focal spots that can be moved together as a pattern, and which will simultaneously accomplish LIOB. Merely moving the focal spot faster, however, may be impractical due to functional limitations of the systems operational components. Thus, as a practical matter, the possibility of using multiple focal spots has more promise.

Beam splitters that will divide a primary laser beam into a plurality of different beams are well known. Specifically, “1 to 3,” “1 to 4” and “1 to 7” beam splitters are commercially available. A common characteristic of these beam splitters, however, is that the resultant pattern of focal spots has a fixed orientation relative to the grating of the beam splitter. With this in mind, it may be desirable to move the pattern of focal spots that is created by the beam splitter along a spiral path for some ophthalmic laser surgical procedures. If so, the fixed orientation of the focal spot pattern relative to the grating can result in an uneven dispersion of LIOB locations relative to the path along which the focal spot pattern is being moved.

With the above in mind, it is an object of the present invention to provide a system and method for dispersing focal spots on a spiral path through a treatment area during ophthalmic laser surgery, with modulated movements to achieve a more homogeneous dispersion of LIOB locations in the treatment area. Another object of the present invention is to provide a system and method for ophthalmic laser surgery with modulated movements of multi-focal spots that include modulations of speed and line spacing. Yet another object of the present invention is to provide a system and method for dispersing focal spots on a spiral path through a treatment area during ophthalmic laser surgery that is relatively easy to manufacture, is simple to operate and is comparatively cost effective.

### SUMMARY OF THE INVENTION

This invention pertains to a system and to a method for moving a laser beam for the Laser Induced Optical Breakdown (LIOB) of tissue in a target area (e.g. the cornea of an eye). More specifically, the present invention concerns the movement of a pattern of focal points that results in the target area when the laser beam is passed through a beam splitter (grating). Importantly, for this invention the beam splitter remains stationary, and an optical scanner is employed to move the laser beam along an essentially spiral path.

In the context of a geometry for an essentially spiral path, the following definitions are appropriate. An angle “θ” identifies the angular position of the scanner (laser beam) at a point in time; “ω” is the angular speed (velocity) of the laser beam; “r” is the radial distance of a point on the path from the center of rotation; and “Δr” is the spacing between adjacent tracks at a same angular position. As envisioned for the present invention, as “θ” changes through an arc of 3600, the distance “r” will also change (e.g. decrease during each rotation). Depending on the type of beam splitter that is being used (e.g. “1 to 3”; “1 to 4”; or “1 to 7”), and the particular requirements of the procedure being followed, the angular speed “c”, and the spacing “Δr” may be varied sinusoidally.

Recall the beam splitter remains stationary. Consequently, as the laser beam is being rotated, the pattern of focal points will continuously change its orientation relative to the spiral path. By way of example, and in order to better visualize this change in pattern orientation, consider a complete 360° rotation of the laser beam. Begin this rotation at a start point “rₒ” on the essentially spiral path, where the angle “θ” is zero (i.e. θ=0° at rₒ). Also, consider a “1 to 3” beam splitter is used to create a linear pattern of three focal points. And, have the beam splitter be positioned to orient the line of three focal points perpendicular to the path, at the start point (i.e. at rₒ).

For the case presented immediately above, after leaving the start point where the pattern is perpendicular to the path, the pattern will sequentially be tangent to the path at θ=90°, perpendicular to the path at θ=180°, again tangent to the path at θ=270°, and again perpendicular to the path back at the start point where θ=0°. The consequence of this is that, for a constant ω and a constant Δr, the tangential orientation of the focal point pattern near θ=90°, 270°, will cause LIOB locations to become more concentrated along the path. This is aggravated by the fact that at these same values for “θ”, LIOB locations become less concentrated in the areas between adjacent tracks of the path. Thus, it may be desirable to move the laser beam at faster angular speeds (i.e. increase ω), and to also diminish the spacing (i.e. decrease Δr) in the vicinities where θ=90°, 270°. The discussion here specifically pertains to LIOB in a plane, such as when the cornea is aplanated. For a normal spherical cornea, however, variations in the z-direction may also need to be accounted for.

With the above in mind, the present invention envisions a phase modulation of the angular speed (velocity) “ω”, as the laser beam (i.e. pattern of focal points) is moved along its path. Additionally, but not necessarily, the spacing between adjacent tracks, “Δr”, can also be phase modulated. In accordance with the present invention, and in both cases (“ω” and “Δr”), phase modulation is employed to achieve a more uniform distribution of focal points for LIOB in the target area. When a “1 to 3” beam splitter is being used, this can be accomplished by moving the pattern faster in the region near θ=90°, 270° while diminishing “Δr”.

Mathematically, phase modulations in accordance with the present invention can be accomplished with the expressions set forth below. For phase modulation of the angular speed:

ω′=ω(1+F sin(nθ))

wherein:

ω′ is the actual phase-modulated angular speed;

ω is a predetermined angular speed (i.e. the angular speed at θ 0°);

F is a magnitude for the phase modulation control;

n is an integer; and

θ is the angular position of the scanner.

For phase modulation of the spacing:

Δr′=Δr(1+f sin(nθ))

wherein:

Δr′ is the actual phase modulated track spacing;

Δr is a predetermined track spacing (i.e. spacing at θ=0°);

f is a magnitude for the phase modulation control;

n is an integer; and

θ is the angular position of the scanner.

And, for phase modulation in the z-direction (i.e. when a domed surface is to be altered by LIOB):

Δz′=Δz(1+f sin(nθ))

wherein:

Δz′ is the actual phase modulated track spacing in the z-direction;

Δz is a predetermined track spacing in the z-direction (i.e. closest spacing in z-direction at θ=0°);

f is a magnitude for the phase modulation control;

n is an integer; and

θ is the angular position of the scanner.

In the above expressions, “F”, “f”, “f” and “n” can be selected, as desired, by the operator. In most instances, as indicated above, the values for these variables will be dependent on the type of beam splitter that is being employed (i.e. “1 to 3”; “1 to 4”; or “1 to 7”).

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a layout of functional components for a surgical laser system in accordance with the present invention;

FIG. 2 is a schematic diagram showing a multi-focal spot pattern that has been created with a “1 to 3” beam splitter, as would be seen along the line 2-2 in FIG. 1, with the orientation of the pattern being shown relative to a spiral path, as the pattern is moved along the path;

FIG. 3 is a schematic diagram showing a focal spot dispersion that results from cyclical pattern lapping during execution of a phase modulated routine in accordance with methods of the present invention; and

FIG. 4 is a schematic diagram showing the progress of a multi-focal spot pattern that has been created by a “1 to 4” beam splitter.

### DESCRIPTION OF THE PREFERRED EMBODIMENTS

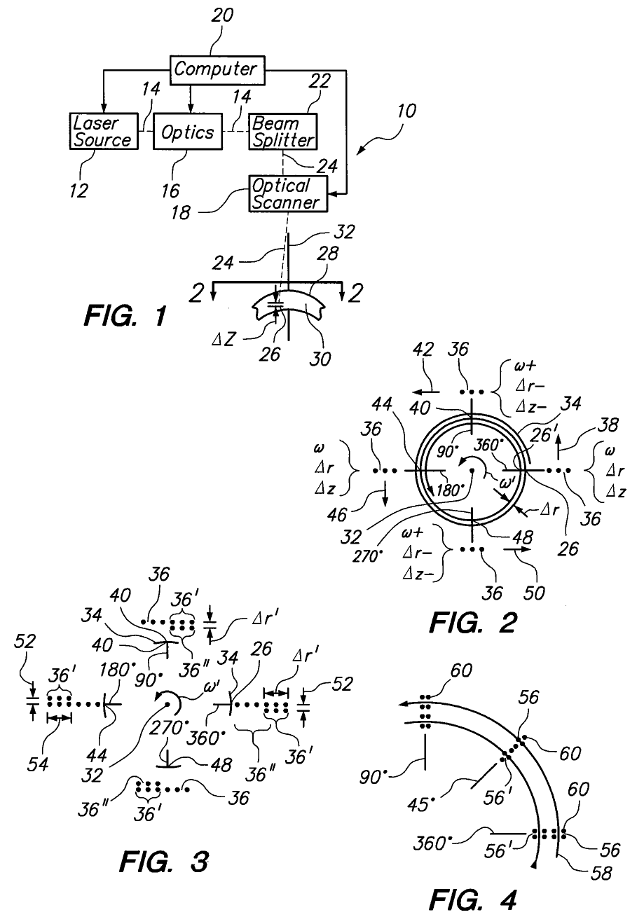

Referring initially to FIG. 1, a system for dispersing focal spots on a spiral path through a treatment area during ophthalmic laser surgery is shown and is generally designated 10. As shown, the system 10 includes a laser source 12 for generating a primary laser beam 14. For purposes of the present invention, the laser source 12 can be of any type well known in the pertinent art that is capable of performing Laser Induced Optical Breakdown (LIOB) during an ophthalmic laser surgery procedure.

FIG. 1 also shows that the system 10 includes optics 16 for focusing the primary laser beam 14, and an optical scanner 18 for moving the focal point of the primary laser beam 14. Further, system 10 includes a computer 20 that coordinates the operations of the optics 16 and optical scanner 18 with that of the laser source 12. System 10 also includes a beam splitter 22 for converting the primary laser beam 14 into a multi-beam 24. As envisioned for the system 10 the beam splitter 22 may be of any type well known in the pertinent art, such as a “1 to 3,” a “1 to 4,” or a “1 to 7” beam splitter. Accordingly, the multi-beam 24 may respectively have a pattern of 3, 4 or 7 focal spots. For discussion purposes hereinafter, the beam splitter 22 will initially be considered to have a “1 to 3” grating.

Still referring to FIG. 1 it is seen that the multi-beam 24 is directed from the beam splitter 22 and then by the optical scanner 18, to a spot 26 in the cornea 28 of an eye. As envisioned by the present invention, the multi-beam 24 will then be moved through a treatment area 30 in the cornea 28 by rotating the multi-beam 24 around a central axis 32 along a substantially spiral path 34 (see FIG. 2). Specifically, it is envisioned that the system 10 will accomplish LIOB over a substantially circular surface area in the cornea 28. This area may be either planar or dome-shaped.

With reference to FIG. 2, it will be seen that the multi-beam 24 actually establishes a pattern 36 of focal points at the spot 26. As illustrated, the pattern 36 is a line of three focal points. Such a pattern 36 is typical for a beam splitter 22 having a “1 to 3” grating. Moreover, due to the alignment of the beam splitter 22 relative to optical scanner 18, the pattern 36 will be oriented substantially perpendicular to the spiral path 34 at the spot 26. At the spot 26, the pattern 36 is rotating around the axis 32 with an angular velocity “ω” and, thus, is traveling in the direction of arrow 38. Because the beam splitter 22 is held stationary, however, the pattern 36 will change its orientation relative to the path 34 as it moves further along the path 34.

In FIG. 2, consider the spot 26 to be a start point where an angle θ relative to the central axis 32 is established as 0° (also 360°). As the pattern 36 rotates around the axis 32, and away from the spot 26 (i.e. in the direction of arrow 38 at spot 26), it will thereafter gradually change its orientation. This change continues until at the spot 40 (θ=90°) the pattern 36 is oriented tangential to the path 34. At this point the pattern 36 is traveling in the direction of arrow 42. Continued movement of the pattern 36 along the path 34 will further reorient the pattern 36 perpendicular to the path 34 at the spot 44 (θ=180°). Its change in direction of travel is now indicated by arrow 46. At the spot 48 (θ=270°), however, the pattern 36 is again tangential to the path 34. Moving away from the spot 48 in the direction of arrow 50 the pattern 36 comes to the spot 26′ where it is again perpendicular to the path 34 (again θ=0°). On the spiral path 34, the spot 26′ is shown offset from the spot 26 by a spacing Δr.

The sequence of rotation as described above poses issues that affect the distribution of focal points for LIOB. If advancement of the pattern 36 along the path 34, i.e. the distance 52 between LIOB episodes on the path 34, is less than the length 54 of the three-spot pattern 36 (see θ=180° in FIG. 3), there can be unwanted overlaps of successive patterns 36. Such overlaps are to be avoided. In particular, this condition will most egregiously occur in the vicinity of spots 40 and 48 (θ=90° and 270°).

With the above in mind, system 10 provides for phase modulation of the movements of pattern 36 as it moves along the path 34 through the treatment area 30 in cornea 28. Mathematically, phase modulations in accordance with the present invention can be accomplished with the expressions set forth below. For phase modulation of the angular speed:

ω′=ω(1+F sin(nθ))

wherein:

ω is the actual phase-modulated angular speed;

ω is a predetermined angular speed (i.e. the angular speed at θ=0°);

F is a magnitude for the phase modulation control;

n is an integer; and

θ is the angular position of the scanner.

The consequence of this phase modulation is shown in FIG. 3.

In FIG. 3 a sequence of patterns 36 are shown during a rotation about the axis 32. Specifically, in this sequence, the pattern 36′ immediately precedes the pattern 36 in LIOB. On the other hand, the pattern 36″ follows the pattern 36 and causes LIOB to occur on the subsequent revolution of patterns around the axis 32. Also, for purposes of disclosure, consider the advancement distance 52 between patterns 36′ and 36 to be approximately 10 microns at spots 26 and 44 (θ=0° and 180°). Also, consider the length 54 of patterns 36, 36′ and 36″ to be approximately forty microns. FIG. 3 then shows that with a proper phase modulation of the angular velocity “ω” around axis 32, the speed of the patterns 36 and 36′ can be controlled to prevent excessive overlap. Specifically, the speed can be established so that the patterns 36 will not adversely overlap in the vicinity of spots 40 and 48 (θ=90° and 270°). Stated differently, the advancement distance 52 has been substantially increased beyond what is established for movement of the pattern 36 at spots 26 and 44 (θ=0° and 180°). For the present invention, these changes in “ω” are controlled by the computer 20 which will cause the optical scanner 18 to function in accordance with phase modulation for “ω” disclosed above.

In addition to phase modulation of the angular speed “ω”, the system 10 also envisions phase modulation for the spacing “Δr” during successive rotations along the spiral path 34. For phase modulation of the spacing:

Δr′=Δr(1+f sin(nθ))

wherein:

Δr′ is the actual phase modulated track spacing;

Δr is a predetermined track spacing (i.e. spacing at θ=0°);

f is a magnitude for the phase modulation control;

n is an integer; and

θ is the angular position of the scanner.

Still referring to FIG. 3, it will be seen that in its subsequent pass by spot 26 (i.e. at spot 26′), the pattern 36″ has been radially offset by the distance Δr′. Using forty microns for the length 54, this Δr′ at spot 26 will preferably be about thirty microns. At spots 40 and 48 (0=90° and 270°), however, a spacing Δr′ of thirty microns would produce an unsatisfactory result. Specifically, it would aggravate the overlap issue discussed above by leaving an overly extended spacing Δr′ between laps of the spiral path 34. Accordingly, by properly using phase modulation for spacing, i.e. by reducing Δr′ at spots 40 and 48 (0=90° and 270°) the patterns 36 will be more evenly distributed.

If it is desired to perform LIOB on a planar surface in the treatment area 30, there is no need to modulate the location of successive patterns 36 in the z-direction. On the other hand, if it is desired to create a dome-shaped surface for LIOB in the treatment area 30, consideration must be given to the z-direction. Further, due to the phase modulation of spacing, Δr′, discussed above, it may be desirable to also phase modulate in the z-direction. If so, for phase modulation in the z-direction (i.e. when a domed surface is to be altered by LIOB):

Δz′=Δz(1+f sin(nθ))

wherein:

Δz′ is the actual phase modulated track spacing in the z-direction;

Δz is a predetermined track spacing in the z-direction (i.e. closest spacing in z-direction at θ=0°);

f is a magnitude for the phase modulation control;

n is an integer; and

θ is the angular position of the scanner.

A somewhat different situation is presented when the beam splitter 22 is employed with a “1 to 4” grating. Unlike the linear, one-dimensional pattern 36 that is presented when a “1 to 3” grating is used, a “1 to 4” grating creates a square, two-dimensional pattern 56 (see FIG. 4). Specifically, as shown in FIG. 4, the pattern 56 has both an azimuthal dimension and a radial dimension. The two-dimensional aspect of the pattern 56, however, has consequences when it is moved along a spiral path 58.

With a “1 to 4” grating held stationary, as the optical scanner 18 moves the multi-beam 24 along a spiral path 58, the resultant pattern 56 will maintain a fixed orientation. The same phenomenon has been considered above with regard to the pattern 36 of a “1 to 3” grating. When using a “1 to 4” grating, however, without some compensation it happens that gaps will develop between focal spots along both the horizontal and vertical axes. Why this happens can be best appreciated with reference to FIG. 4.

In FIG. 4 a portion of a spiral path 58 is shown between its 360° (i.e. 0°) position and the 90° position. Using focal spot 60 in this pattern 56 for orientation purposes, it will be seen that as the pattern 56 is moved along the path 58, it appears to rotate on the path 58. A consequence of this apparent rotation is that on subsequent passes (e.g. shown by pattern 56′ in FIG. 4) the focal spots for LIOB tend to concentrate or “bunch up” at the 45° position. Attempts to alleviate the adverse consequence of concentrated focal spots by increasing the speed of the pattern 56 between the horizontal axis (i.e. 0° or 360° position) and the vertical axis (i.e. 90° position) will then create gaps along these axes.

Aside from the issues noted above, when compared with a single focal spot system, it would seem a “1 to 4” beam splitter 22 can increase the speed of a procedure four fold. The phenomenon mentioned above, however, diminishes the practicality of this possibility. Instead, it can be geometrically shown that using smaller separations between spots in the pattern 56, in combination with phase modulation, is useful for increasing the homogeneity of LIOB spot locations. Accordingly, as envisioned for the present invention, spot separation in the pattern 56 of approximately seven microns, and a phase modulation using the expression ω′=ω(1+F sin 2θ) appears optimal. The result is a scanning procedure that can be accomplished in about half the time otherwise required for a single spot system.

As intended for the present invention, the computer 20 is programmed with the desired phase modulation routines. In each instance, the variables “F”, “f” and “f” may be appropriately selected to suit the particular needs of the surgical procedure.

While the particular System and Method for Photoablation Using Multiple Focal Points Using Angular Phase Modulation as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for dispersing focal spots on a spiral path through a treatment area during ophthalmic laser surgery which comprises:
a source for generating a primary laser beam;
a means for splitting the primary laser beam into a plurality of secondary laser beams;
an optical means for focusing the plurality of secondary laser beams into a pattern of respective focal points;
a scanning means for moving the pattern of focal points along the spiral path; and
a computer means connected to the scanning means for moving the pattern of focal points along the spiral path in accordance with a routine having a phase modulated angular velocity.

2. A system as recited in claim 1 wherein the routine is defined by the phase-modulated relationship
ω′=ω(1+F sin(nθ))
where ω′ is an angular speed after phase modulation, ω is an original angular speed, F is a magnitude factor for phase modulation control, n is an integer, and θ is an angular position.

3. A system as recited in claim 2 wherein the routine is further defined by the phase-modulated relationship
Δr′=Δr(1+f sin(nθ))
where Δr′ is a line spacing after phase modulation, Δr is an original line spacing, and f is a magnitude factor for phase modulation control.

4. A system as recited in claim 3 wherein “rₒ” is the radius of the spiral path when θ=0° and wherein “rₒ” changes in a range from about 4.5 mm to approximately 0.5 mm during a routine.

5. A system as recited in claim 3 wherein the routine is defined by the phase-modulated relationship
Δz′=Δz(1+f sin(nθ))
where Δz′ is spacing in a z-direction after phase modulation, Δz is an original spacing, and f is a magnitude factor for phase modulation control.

6. A system as recited in claim 1 wherein the splitting means is a grating.

7. A system as recited in claim 1 wherein the splitting means is a one to three grating.

8. A system as recited in claim 1 wherein the scanning means is a plurality of galvo mirrors.

9. A system as recited in claim 1 wherein the primary laser beam is a pulsed laser beam having an energy level variable in an approximate range between 1.5 μJ and 9 μJ, with a pulse duration less than one picosecond, and a pulse interval of approximately 25 μsec.

10. A system for dispersing focal spots on a spiral path through a treatment area during ophthalmic laser surgery which comprises:
a means for focusing a plurality of laser beams to create a pattern of focal spots;
a means for moving the pattern of focal spots along a predetermined path through a target area for performing laser induced optical breakdown (LIOB) of tissue at sequential LIOB locations in the target area; and
a means for varying the speed of the pattern along the path to achieve a substantially homogeneous dispersion of the LIOB locations.

11. A system as recited in claim 10 wherein the focusing means comprises:
a source for generating a primary laser beam;
a means for splitting the primary laser beam into a plurality of secondary laser beams; and
an optical means for focusing the plurality of secondary laser beams into a pattern of respective focal points.

12. A system as recited in claim 11 wherein the moving means and varying means are incorporated into a computer means to move the pattern along a spiral path in accordance with a routine.

13. A system as recited in claim 12 wherein the routine is defined by the phase-modulated relationship
ω′=ω(1+F sin(nθ))
where ω′ is an angular speed after phase modulation, ω is an original angular speed, F is a magnitude factor for phase modulation control, n is an integer, and θ is an angular position.

14. A system as recited in claim 13 wherein the routine is further defined by the phase-modulated relationship
Δr′=Δr(1+f sin(nθ))
where Δr′ is a line spacing after phase modulation, Δr is an original line spacing, and f is a magnitude factor for phase modulation control.

15. A system as recited in claim 14 wherein the routine is defined by the phase-modulated relationship
Δz′=Δz(1+f sin(nθ))
where Δz′ is spacing in a z-direction after phase modulation, Δz is an original spacing, and f is a magnitude factor for phase modulation control.

16. A system as recited in claim 10 wherein the focusing means includes a beam splitter having a “1 to 3” grating.

17. A method for dispersing focal spots on a spiral path through a treatment area during ophthalmic laser surgery which comprises the steps of:
generating a primary laser beam;
splitting the primary laser beam into a plurality of secondary laser beams;
focusing the plurality of secondary laser beams into a pattern of respective focal points;
scanning the pattern of focal points along the spiral path; and
moving the pattern of focal points along the spiral path in accordance with a routine having a phase modulated angular velocity.

18. A method as recited in claim 17 wherein the routine is defined by the phase-modulated relationship
ω′=ω(1+F sin(nθ))
where ω is an angular speed after phase modulation, ω is an original angular speed, F is a magnitude factor for phase modulation control, n is an integer, and θ is an angular position.

19. A method as recited in claim 18 wherein the routine is further defined by the phase-modulated relationship
Δr′=Δr(1+f sin(nθ))
where Δr′ is a line spacing after phase modulation, Δr is an original line spacing, and f is a magnitude factor for phase modulation control.

20. A method as recited in claim 19 wherein the routine is further defined by the phase-modulated relationship
Δz′=Δz(1+f sin(nθ))
where Δz′ is spacing in a z-direction after phase modulation, Δz is an original spacing, and f is a magnitude factor for phase modulation control.
